(19) United States
(12) Patent Application Publication
(10) Pub. No.: US 2010/0114103 A1
(43) Pub. Date: May 6, 2010

(54) APPARATUS AND METHODS FOR ALTERATION OF ANATOMICAL FEATURES

(75) Inventors: Michael R. Harrison, San Francisco, CA (US); Richard J. Fechter, San Rafael, CA (US); Arthur Moran, San Bruno, CA (US); Darrell Christensen, Petaluma, CA (US)

Correspondence Address: JOHN P. O'BANION;O'BANION & RITCHEY LLP, 400 CAPITOL MALL SUITE 1550, SACRAMENTO, CA, 95814, US

(73) Assignee: THE REGENTS OF THE UNIVERSITY OF CALIFORNIA, Oakland, CA (US)

(21) Appl. No.: 12/266,485

(22) Filed: Nov. 6, 2008

(51) Int. Cl. A61B 17/58; A61F 2/44

(52) U.S. Cl. 606/90; 623/17.16; 623/17.11

(57) ABSTRACT

Systems and methods are disclosed for manipulating an anatomical feature within the body of the patient. An implant such as an internal jackscrew is implanted at the anatomical and has first and second attachment points that secure to spaced-apart locations on the anatomical feature. An internal motor is coupled to the jackscrew, and is configured to drive motion of the jackscrew to manipulate the anatomical feature. The system further includes an external driver that is inductively coupled to the internal motor to manipulate the anatomical feature.

### CROSS-REFERENCE TO RELATED APPLICATIONS

This application is related to U.S. Published Application No. 2006/0271107, published on Nov. 30, 2006, incorporated herein by reference in its entirety, and to U.S. Published Application No. 2006/0074448, published on Apr. 6, 2006, incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND OF THE INVENTION

1. Field of the Invention

This invention pertains generally to apparatus and methods for incrementally manipulating body structures and more particularly to performing corrective procedures on a patient via incremental internal loading.

2. Description of Related Art

Anatomical deformities occur in the general populous in a number of different forms and from a variety of causes. Examples of skeletal deformities include pectus excavatum, scoliosis, club feet, and numerous forms of skeletal dysplasia. These conditions are treated in a variety of different manners from braces to surgery, with sometimes minimal efficacy.

The defect known as pectus excavatum, or funnel chest, is a congenital anomaly of the anterior chest wall. The excavatum defect is characterized by a deep depression of the sternum, usually involving the lower half or two thirds of the sternum, with the most recessed or deepest area at the junction of the chest and the abdomen. The lower 4-6 costal or rib cartilages dip backward abnormally to increase the deformity or depression and push the sternum posterior or backward toward the spine. Also, in many of these deformities, the sternum is asymmetric or it courses to the right or left in this depression. In many instances, the depression is on the right side.

Pectus excavatum with significant deformity occurs in approximately 1 out of every 2000 births. The deformity may be present at birth but is often noted after several years of age and usually worsens during rapid growth around puberty. Because of the pressure of the sternum and cartilages, defect also pushes the midline structures so that the lungs are compressed from side to side and the heart (right ventricle) is compressed. Severe lesions have a major effect on thoracic volume and pulmonary function but the principal motivation for repair is the deformity itself. It does occur in families and thus, is inherited in many instances. Other problems, especially in the muscle and skeletal system, also may accompany this defect. In approximately ⅕ of the patients, scoliosis is present. The regression or any improvement in this defect rarely occurs because of the fixation of the cartilages and the ligaments. When one takes a deep breath or inspires, the defect is usually accentuated.

Pectus excavatum can be repaired surgically using an open approach in which the malformed costal cartilages are resected and the sternum forcibly held in place with a metal strut. In another approach, described in U.S. Pat. No. 6,024,759, the sternum is forced into a corrected position often under great tension, and held in place with a metal strut. Both can achieve good results but at the cost of considerable morbidity: an operation under general anesthesia followed by a 4-7 day hospital stay required for pain control usually by continuous epidural analgesia. Several more weeks of moderate to severe discomfort are typical and complications from the sternum held forcibly against the metal strut are not infrequent. It is necessary to leave the bar in place for a year or more before it is removed in another procedure. Total cost usually reimbursed by third party payers averages more than $30,000.

The problem with all currently available pectus excavatum surgical repairs is that they attempt to achieve immediate total correction and fixation often under considerable tension. A better approach would be the gradual step-by-step correction of the deformity by applying a smaller force over a longer period of time.

Another skeletal deformity, scoliosis, is a condition in which an individual has an abnormal spine curvature. Generally, some curvature in the neck, upper trunk and lower trunk is normal. However, when there are abnormal side-to-side (lateral) curves in the spinal column, the patient is generally diagnosed as having as scoliosis.

Orthopaedic braces are typically used to prevent further spinal deformity in children with curve magnitudes within the range of 25 to 40 degrees. If these children already have curvatures of these magnitudes and still have a substantial amount of skeletal growth left, then bracing is a viable option. The intent of bracing, however, is to prevent further deformity, and is generally not used to correct the existing curvature or to make the curve disappear.

Surgery is an option used primarily for severe scoliosis (curves greater than 45 degrees) or for curves that do not respond to bracing. The two primary goals for surgery are to stop a curve from progressing during adult life and to diminish spinal deformity.

Although there are different techniques and methods used today for scoliosis surgery, all of them involve fairly invasive procedures with considerable patient morbidity. One frequently performed surgery involves posterior spinal fusion with instrumentation and bone grafting, which is performed through the patient's back. During this surgery, the surgeon attaches a metal rod to each side of the patient's spine by anchors attached to the vertebral bodies. The spine is then fused with a bone graft. The operation usually takes several hours and the patient is typically hospitalized for a week or more. Most patients are not able to return to school or for several weeks after the surgery and cannot perform some pre-operative activities for up to four to six months.

Another surgery option for scoliosis is an anterior approach, wherein the surgery is conducted through the chest walls instead of entering through the patient's back. During this procedure, the surgeon makes incisions in the patient's side, deflates the lung and removes a rib in order to reach the spine. The anterior spinal approach generally has quicker patient rehabilitation, but usually requires bracing for several months after this surgery.

For these reasons, it would be desirable to provide improved apparatus and methods for repositioning bone structures, by applying a corrective force to the bone structure, which could be gradually adjusted much like orthodontic tooth braces.

It would be further desirable to provide a device that applies a corrective force to reposition a body member without a mechanical force that requires piercing of the skin, thereby limiting the specter of infection and wound problems.

In addition, it would be desirable to provide a device for repositioning bones structures having tension-sensing technology to allow measurement of the force applied to correct all types of asymmetric deformities and allow protection of skin against pressure damage.

In addition, it would be desirable to provide improved devices and methods for minimally invasively treating scoliosis.

At least some of these objectives will be met with the inventions described hereinafter.

### BRIEF SUMMARY OF THE INVENTION

The present invention comprises apparatus and methods for altering the position, orientation, growth or development of body parts and organs by sustained force over time.

The present invention comprises an implantable jackscrew that is non-invasively activated, lengthened, or shortened, via an induced electrical coupling across the skin. The entire implanted jackscrew device is hermetically sealed within an expandable titanium bellows. The jackscrew is driven by an electric motor within the screw device. The electric motor is connected to a subcutaneous docking station. The small electric motor may comprise a piezo motor or any other available small electric motor capable of generated forces up to 100 lbs that can be activated non-invasively from outside the skin using inductive power and signal coupling. The external device supplies the power and displays the force and distance readings from the implanted device.

The implanted device incorporates a force measurement transducer. For some conditions that would benefit from a “cushioned” application of force, a coil spring shock absorber using either magnetic repulsion or an elastomer spring may be used.

An aspect of the invention is an apparatus for incrementally adjusting the length between a first body segment and a second body segment within the body of a patient. The apparatus includes an implant configured to be installed within the body having a first member with a first attachment point for fixation to the first body segment, and a second member with a second attachment point for fixation to the second body segment. The first member is moveably coupled to the second member to allow linear motion of the first member with respect to the second member. The apparatus further includes a motor coupled to the first and second members, wherein the first member is coupled to the second member via a worm drive such that rotation of the motor drives motion of the worm drive to affect translation of the second member with respect to the first member. The electronic motor is transcutaneously coupled to a power source external to the patient's body and the motor is configured to rotate in response to energy delivered from the power source to incrementally adjust the length between the first attachment point and the second attachment point.

In one embodiment, the unit comprises a gear reduction unit coupled between the motor and the worm drive, wherein the gear reduction unit facilitates a high ratio gear reduction of the rotation of the first rotor to the worm drive.

In a preferred embodiment, the motor is inductively coupled to the power source.

In another embodiment, the power source comprises a control to vary the speed and directionality of the internal motor to allow micro-motion control of the distance between the first and second attachment points.

The apparatus may also include a force measurement transducer coupled to the first or second members, wherein the transducer is configured to measure a force applied to the first and second attachment points by the implant. Readings from the transducer may provide feedback for control of the internal motor.

Furthermore, a biasing member may be coupled to the first or second members; wherein the biasing member is configured to absorb loading between the first and second members.

In one embodiment, the first attachment point is configured to secure to a first vertebra and the second attachment point is configured to attach to a second vertebra, wherein the implant is configured to distract the first vertebra from the second vertebra.

In a preferred embodiment, the internal motor, worm drive, and first and second members are hermetically sealed inside a casing.

Another aspect is a method for manipulating first and second body segments within the body of a patient by inserting an implant at a location within the body, securing a first attachment point of the implant to the first body segment, securing a second attachment point of the implant to the second body segment, and transcutaneously supplying power to an internal motor coupled to the first and second attachment points. The internal motor provides rotation to a worm drive coupled between the first and second attachment points such that the worm drive transforms the rotational motion of the internal rotor into linear adjustment of the distance between the first and second attachment points.

In a preferred embodiment of the current aspect, adjusting the distance between the first and second attachment allows incremental manipulation of the first body segment with respect to the second body segment.

In another embodiment, a first member comprising the first attachment point is moveably coupled to a second member comprising the second attachment point, and adjusting the distance between the first and second attachment points comprises linearly translating the first member with respect to the second member. The gear ratio between the internal motor and the worm drive may be reduced to allow a smaller input force on the internal motor to drive a larger output force between the first and second attachment points.

The method may further include controlling the speed and directionality of the internal motor rotation to affect micro-motion control of the distance between the first and second attachment points.

In another embodiment, the method includes measuring a force applied to the first and second body segments by the implant. The force measurement may be wirelessly transmitted the force measurement to a controller external to the patient to control the internal motor according to feedback provided by the force measurements.

In yet another embodiment, the method may include preloading the first and second attachment points by coupling a biasing member to the first or second members.

In a preferred embodiment, transcutaneously supplying power to an internal motor comprises inductively transferring energy from an external location to a subcutaneous location within the patient.

In one embodiment, the first segment comprises a first vertebrae of the spine and the second segment comprises a second vertebrae of the spine, wherein the first attachment point is secured to the first vertebrae and the second attachment point is secured to the second vertebra so that motion of the first and second attachment points distracts the first vertebrae from the second vertebrae.

Another aspect is a system for manipulating an anatomical feature within the body of the patient comprising an internal jackscrew configured to be implanted at the anatomical feature inside the patient. The jackscrew comprises first and second attachment points configured to secure to spaced-apart locations on the anatomical feature. An internal motor is coupled to the jackscrew, wherein the internal motor is configured to drive motion of the jackscrew to manipulate the anatomical feature. The system further includes a controller configured to supply energy to the internal motor, wherein the controller is located external to the patient. An inductive coupling is connected to the controller and internal motor and is configured to wirelessly transfer energy from the external controller to the internal motor.

In one embodiment, the inductive coupling comprises an external pad coupled to the controller; and an internal pad coupled to the internal motor, wherein the internal pad is configured to be positioned at a subcutaneous location to wirelessly transmit energy from the controller through the skin to the internal motor.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only, and where like reference numbers denote like elements:

FIG. 1 is a schematic view of an internal jackscrew assembly in accordance with the present invention.

FIG. 2 shows an alternative embodiment in accordance with the present invention.

FIG. 3 shows the embodiment of FIG. 1 installed to decompress a spine segment in accordance with the present invention.

FIG. 4A is an anterior view of the human spine.

FIG. 4B is a lateral view of the human spine.

FIG. 5A-D illustrates various abnormal curvatures of the spine due to scoliosis.

FIG. 6 illustrates abnormal rotation of the vertebrae of the spine as a result of scoliosis.

### DETAILED DESCRIPTION OF THE INVENTION

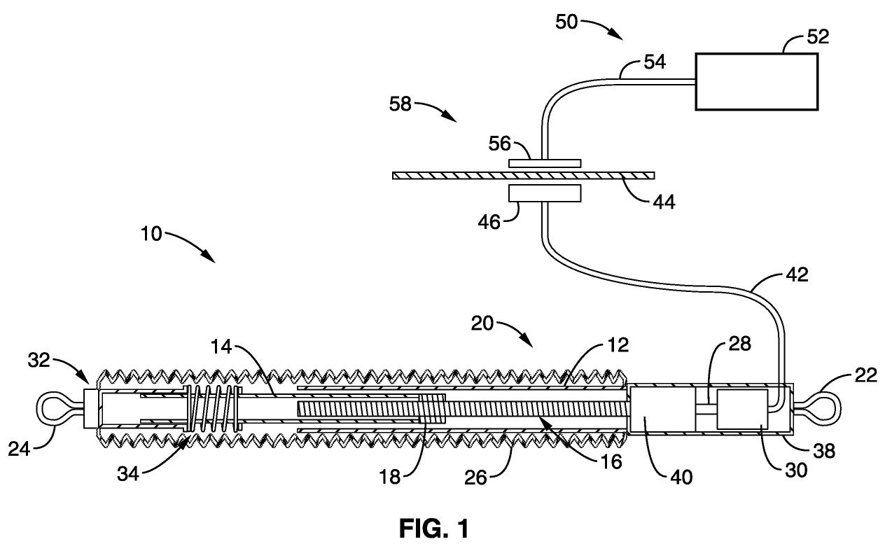

Referring more specifically to the drawings, for illustrative purposes the present invention is embodied in the apparatus and methods generally shown in FIG. 1 through FIG. 6. It will be appreciated that the apparatus may vary as to configuration and as to details of the parts, and that the methods may vary as to the specific steps and sequence, without departing from the basic concepts as disclosed herein.

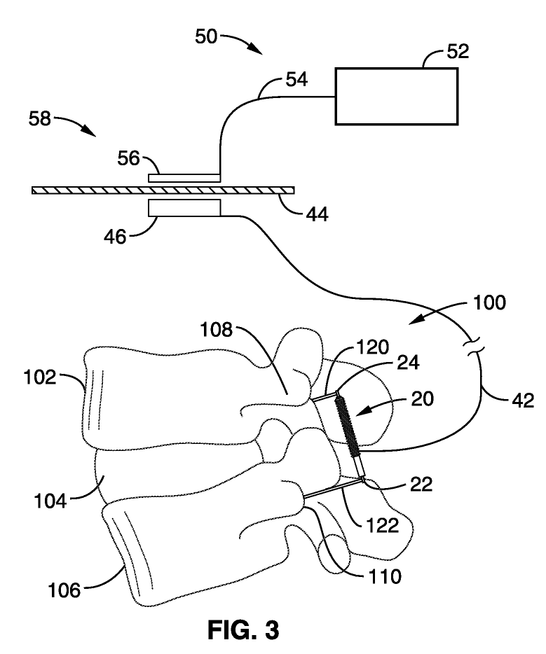

FIG. 1 shows an internal load generating system 10 in accordance with the present invention. The system 10 includes a magnetically coupled implantable jackscrew assembly 20 that is inductively driven by an external drive assembly 50. The jackscrew assembly 20 comprises a first member 12 and second member 14 housed within a hermetically sealed bellows 26. The first and second members 12, 14 are coupled to allow linear motion with respect to each other to apply a tensile or compressive force to respective attachment points 22 and 24 that may be attached to one or more body members or body member locations. For example, attachment point 22 may be coupled to a first vertebral body, and attachment point 24 may be coupled to a second vertebral body to allow incremental distraction of the spine segments (see FIG. 3).

The first member 12 is coupled to an internal drive coupling an electric motor 30. The internal motor 30 is coupled to drive shaft 28 located inside end cap 38. The small electric motor 30 could be a piezo-electric motor or any other available small electric motor capable of generated forces up to 100 lbs or more. The internal motor may comprise any type of rotary or servomotor, including a brush motor or brushless motor.

The internal motor is controlled and powered trans-dermally via an inductive electrical coupling 58 that is configured to wirelessly transfer energy from an external pad 56 to an internal pad or dock 46. Internal induction pad is coupled to the internal motor 30 via cable 42, and is preferably located subcutaneously just ender the patient's skin 44 for optimal transmission. However, other locations in the body may be used as well. The external induction pad 56 is configured to be positioned adjacent or touching the patient's skin 44 just outside the internal pad 46, the location of which may be marked for ease of use.

Inductive coupling 28 may comprise one of several electromagnetic resonant systems available in the art, including dielectric disks or capacitively-loaded conducting-wire loops for pads 56 and 46. The electrical coupling 58 is connected via cable 54 to a power source 52 that supplies the power to the internal motor 30. Power source 52 may also comprise a controller that controls operation of the internal motor 30 (e.g. by operating motor 30 at intervals or according to some other feedback such as that generated by sensor 32).

Sensor 32 may comprise a force measurement transducer that measures the force applied to the attachment points 22, 24. Transducer 32 may be configured to take readings of the applied force over time, and may be configured to store them locally on a memory chip or the like, or transmit force data via the wireless transmission coupling 58 to external receiving unit 52, or may transmit via another wireless remote transmission such as RFID, IR or the like. Transducer 32 may also comprise deformable silicon pressure sensing device, such as the Micro Electro Mechanical Systems (MEMS) implant currently be developed by OrthoMEMS, Inc. for orthopedic sensing.

Poser source/control unit 52 may also comprise a display and user interface to display force and distance readings from sensor 32, and for allowing the force and control settings to be modified.

The rotating shaft 28 coupled to internal motor 30 may also be coupled to gear reduction unit 40 that facilitates a high ratio gear reduction (e.g. 256:1 or 500:1) to worm gear screw 16. Gear reduction unit 40 allows high-speed micro-motion control of the jackscrew assembly 20 via a small input or rotational force from the internal motor 30. The gear reduction unit 40 may comprise a commercially available unit such as Spur Gearhead GS12A or Micro Harmonic Drive MHD 8, both from Maxon Precision Motors, Inc., Fall River, Mass.

Female screw thread or nut 18 is attached to second member 14 and is threaded to screw 16 such that rotation of screw 16 causes the first member 12 to separate or converge with respect to second member from 14. Additional force and separation may be achieved by further rotation of internal motor 30.

The second member 14 may optionally be spring loaded (e.g. via a coil spring, elastomer, or the like) with biasing member 34 to create an additional preload between the first and second members. Biasing member 34 may provide a shock absorption component to the assembly for withstanding loading between first and second body members disposed on attachment points 22 and 24. Initial loading to separate attachment points 24 and 22 may soak up some or all of the travel of biasing member 34, depending on the spring rate. However, as the body members associated with attachments points 24 and 22 are gradually manipulated, the travel of biasing member 34 is restored.

FIG. 1 depicts a linear coil-spring design for biasing member 34. However it is contemplated that an elastomer or magnetic repulsion may also be used.

The entire implanted device is preferably hermetically sealed via endcap 38 and titanium bellows 26 over the moveable members 14, 16.

Pressure applied by the device (either compressive or tensile) is measurable and adjustable through the electric coupling 58 and data provided by sensor 32.

In a preferred embodiment, the jackscrew 20 is operated to provide non-invasive lengthening and shortening in very small increments (i.e., <1 mm), wherein adjustment may be achieved in an awake patient as an out-patient office procedure. This has the advantage of allowing feedback from the patient about patient discomfort or pain relief.

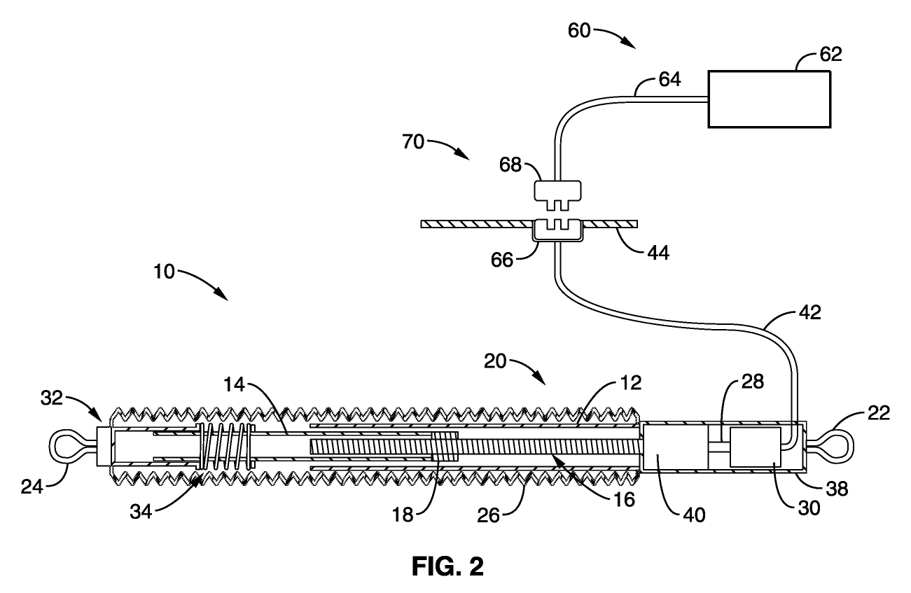

In an alternative embodiment shown in FIG. 2, the internal motor 30 is coupled to controller 62 via a detachable wired coupling 70. In this configuration, the internal motor 30 is coupled to a trans-cutaneous dock 68 that mounts through the skin (e.g. small incision). An external coupling 68 is wired to the controller 62 via cabling 64, and detachably mates with the dock 66 to allow energy and/or data transfer. The monitor/controller 62 may be detached when not in used by separating the external coupling 68 from the dock 66.

1. Vertebral Jack for Decompression of Herniated Disks

FIG. 3 illustrated system 100 for decompression of one or more spine segments. As shown in FIG. 3, a jackscrew assembly 20 may be coupled between vertebra 102 and vertebra 104. In this embodiment, the first attachment 22 is coupled to a pedicle screw 122 that is mounted in the pedicle 110 of the lower vertebra 106. Correspondingly, the second attachment 24 is coupled to a pedicle screw 124 that is mounted in the pedicle 108 of the upper vertebra 102. The jackscrew may then be operated via control unit 52 and inductive coupling 58 to increase the distance between attachment points and thereby place the vertebral joint in tension to leave compression of disc 104 the may be collapsed or herniated.

The pedicle mounting may comprise a number of different systems available in the art, including, fur example, any of the systems are disclosed in U.S. Pat. Nos. 6,648,915; 6,010,503; 5,946,760; 5,863,293; 4,653,481, etc., the entire disclosures of which are incorporated herein by reference.

While FIG. 3 illustrates decompression of adjacent spine members, it is appreciated that the jackscrew assembly 20 may be sized to span any number of vertebrae. In addition, the jackscrew assembly 20 may be mounted anteriorly (e.g. to the vertebral body) or laterally (in which case two jacks may be used for to maintain symmetry).

2. Vertebral Jack for Scoliosis

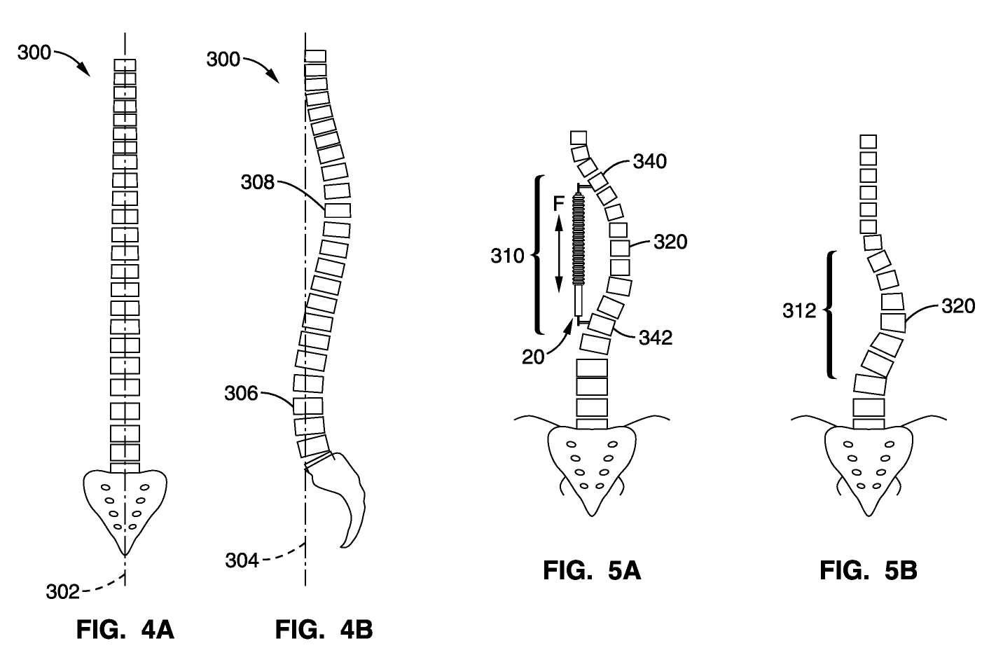

FIGS. 4A and 4B illustrate the curvature of a normal spine 300. The spine is relatively straight in the sagittal plane 302 and has a double curve in the coronal plane 304. Generally, the thoracic section 308 of the spine is convex posteriorly and the lumbar section 306 of the spine is convex anteriorly. Normally there should be no lateral curvature of the spine about the saggital plane 302.

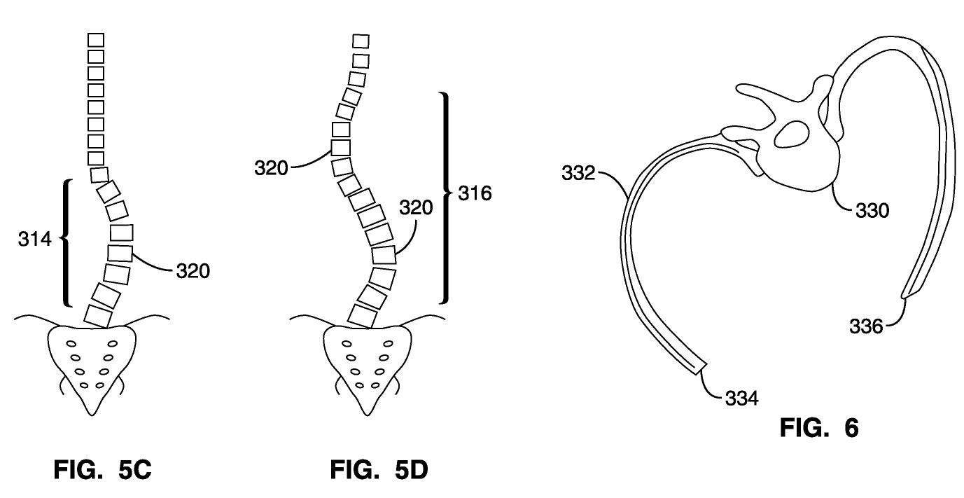

Scoliosis is a deformity that generally comprises by both lateral curvature and vertebral rotation. FIGS. 5A-D illustrate various forms of abnormal lateral curvature of the spine. FIG. 5A shows abnormal thoracic curvature 310. FIG. 5B shows abnormal thoracolumbar curvature 312. FIG. 5C shows abnormal lumbar curvature 314. Finally, some cases involve a double curvature of the spine, as shown in FIG. 5D shows abnormal thoracic curvature.

FIG. 6 illustrates rotation of the spine and corresponding effect on the rib cage 332 s a result of scoliosis. As the disease progresses, the vertebrae 330 and spinous processes in the area of the major curve rotate toward the concavity of the curve. As the vertebral bodies rotate, the spinous processes deviate more and more to the concave side and the ribs follow the rotation of the vertebrae. The posterior ribs on the convex side 336 are pushed posteriorly, causing narrowing of the thoracic cage and the characteristic rib hump seen in thoracic scoliosis. The anterior ribs on the concave side 334 are pushed laterally and anteriorly.

Now referring to FIG. 5A, a jackscrew assembly 20 in accordance with the present invention may be positioned to attach to vertebral segments spanning abnormal thoracic curvature 310. In this configuration, the jackscrew may be expanded to apply a tensile translational force F to the curved section 310 and allow straightening of the intermediary segments and lateral curvature of the spine. The force F may be incrementally applied to continue translation of the vertebrae 340 and 342 over time.

The jackscrew assembly 20 may also be applied to correct for thoracolumbar curvature 312 in FIG. 5B, and lumbar curvature 314 shown in FIG. 5C. Two jackscrew assemblies 20 may be applied to opposite sides of the spine to correct for the double curvature 316 of the spine in FIG. 5D.

Additionally, in any of the conditions shown in FIG. 5A-D, a second opposing jack screw assembly 20 may be attached to the opposing (convex) side of the curvature to and operated to shorten the distance between attachment points and further facilitate curvature correction.

3. Other Applications

The jackscrew assembly 20 scalable to operate under a number of applications. The internal electric motor 30 is available in extremely small sizes, without compromising the output power. The total length of the jackscrew assembly 20 (between attachment points 24 and 22) may range from as little as 1 cm and as great as 30 cm. This allows application to many different diseases and/or conditions. In addition, multiple jackscrew devices 20 can be used and activated individually without interfering with each other or an adjacent device(s).

Accordingly, it is appreciated that the system and methods of the present invention may be used for a variety of applications throughout the body. For example, the system 10 may be used for bone and cartilage elongation and reformation (e.g., distraction osteogenesis), bone lengthening (e.g., leg lengthening), chest deformity correction and chest expansion (e.g., automated titanium rib treatment for thoracic deformities), or adjustment of flow rate, (increase and decrease), through implanted valves (e.g., drug delivery pumps, IV access, shunts, etc.)

Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. An apparatus for incrementally adjusting the length between a first body segment and a second body segment within the body of a patient, comprising:
an implant configured to be installed within the body;
the implant having a first member with a first attachment point for fixation to the first body segment, and a second member with a second attachment point for fixation to the second body segment;
wherein the first member is moveably coupled to the second member to allow linear motion of the first member with respect to the second member; and
a motor coupled to the first and second members;
wherein the first member is coupled to the second member via a worm drive;
wherein rotation of the motor drives motion of the worm drive to affect translation of the second member with respect to the first member;
wherein the electronic motor is transcutaneously coupled to a power source external to the patient's body;
wherein the motor is configured to rotate in response to energy delivered from the power source to incrementally adjust the length between the first attachment point and the second attachment point.

2. An apparatus as recited in claim 1, further comprising a gear reduction unit coupled between the motor and the worm drive.

3. An apparatus as recited in claim 2, wherein the gear reduction unit facilitates a high ratio gear reduction of the rotation of the first rotor to the worm drive.

4. An apparatus as recited in claim 1, wherein the motor is inductively coupled to the power source.

5. An apparatus as recited in claim 4, wherein the power source comprises a control to vary the speed and directionality of the internal motor to allow micro-motion control of the distance between the first and second attachment points.

6. An apparatus as recited in claim 5, further comprising a force measurement transducer coupled to the first or second members;
wherein the transducer is configured to measure a force applied to the first and second attachment points by the implant.

7. An apparatus as recited in claim 6, wherein readings from the transducer provide feedback for control of the internal motor.

8. An apparatus as recited in claim 1, further comprising:
a biasing member coupled to the first or second members;
wherein the biasing member is configured to absorb loading between the first and second members.

9. An apparatus as recited in claim 1:
wherein the first attachment point is configured to secure to a first vertebra and the second attachment point is configured to attach to a second vertebra; and
wherein the implant is configured to distract the first vertebra from the second vertebra.

10. An apparatus as recited in claim 1, wherein the internal motor, worm drive, and first and second members are hermetically sealed inside a casing.

11. A method for manipulating first and second body segments within the body of a patient, comprising:
inserting an implant at a location within the body;
securing a first attachment point of the implant to the first body segment;
securing a second attachment point of the implant to the second body segment;
transcutaneously supplying power to an internal motor coupled to the first and second attachment points;
wherein the internal motor provides rotation to a worm drive coupled between the first and second attachment points;
wherein the worm drive transforms the rotational motion of the internal rotor into linear adjustment of the distance between the first and second attachment points.

12. A method as recited in claim 11, wherein adjusting the distance between the first and second attachment allows incremental manipulation of the first body segment with respect to the second body segment.

13. A method as recited in claim 11, wherein a first member comprising the first attachment point is moveably coupled to a second member comprising the second attachment point; and
wherein adjusting the distance between the first and second attachment points comprises linearly translating the first member with respect to the second member.

14. A method as recited in claim 13, further comprising:
reducing the gear ratio between the internal motor and the worm drive.

15. A method as recited in claim 14, wherein said gear reduction allows a smaller input force on the internal motor to drive a larger output force between the first and second attachment points.

16. A method as recited in claim 13, further comprising:
controlling the speed and directionality of the internal motor rotation to affect micro-motion control of the distance between the first and second attachment points.

17. A method as recited in claim 13, further comprising:
measuring a force applied to the first and second body segments by the implant.

18. A method as recited in claim 17, further comprising:
wirelessly transmitting said force measurement to a controller external to the patient; and
controlling the internal motor according to feedback provided by said force measurements.

19. A method as recited in claim 11, further comprising:
preloading the first and second attachment points by coupling a biasing member to the first or second members.

20. A method as recited in claim 11, wherein transcutaneously supplying power to an internal motor comprises inductively transferring energy from an external location to a subcutaneous location within the patient.

21. A method as recited in claim 11:
wherein the first segment comprises a first vertebrae of the spine and the second segment comprises a second vertebrae of the spine;
wherein the first attachment point is secured to the first vertebrae and the second attachment point is secured to the second vertebra; and
wherein motion of the first and second attachment points distracts the first vertebrae from the second vertebrae.

22. A system for manipulating an anatomical feature within the body of the patient, comprising:
an internal jackscrew configured to be implanted at the anatomical feature inside the patient;
wherein said jackscrew comprises first and second attachment points configured to secure to spaced-apart locations on the anatomical feature;
an internal motor coupled to the jackscrew;
wherein said internal motor is configured to drive motion of the jackscrew to manipulate the anatomical feature;
a controller configured to supply energy to the internal motor;
the controller located external to the patient; and
an inductive coupling configured to wirelessly transfer energy from the external controller to the internal motor.

23. A system as recited in claim 22, wherein the inductive coupling comprises an external pad coupled to the controller; and
an internal pad coupled to the internal motor;
wherein the internal pad is configured to be positioned at a subcutaneous location to wirelessly transmit energy from the controller through the skin to the internal motor.

24. A system as recited in claim 22:
wherein the anatomical feature comprises the patient's spine;
wherein the first attachment point is configured to secure to a first vertebra and the second attachment point is configured to attach to a second vertebra of the spine; and
wherein the jackscrew is configured to incrementally distract the spine.
